(19) United States
(12) Patent Application Publication
(10) Pub. No.: US 2009/0157195 A1
(43) Pub. Date: Jun. 18, 2009

(54) FLUORINATED IMPLANT

(75) Inventor: Gabriel Siedle, Freiburg (DE)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Aesculap AG, a corporation of Germany, Tuttlingen/Donau (DE)

(21) Appl. No.: 12/333,480

(22) Filed: Dec. 12, 2008

(30) Foreign Application Priority Data
Dec. 14, 2007 (DE) 102007062273.4

(51) Int. Cl. A61F 2/04

(52) U.S. Cl. 623/23.74

(57) ABSTRACT

This disclosure relates to a medical implant composed of polymers which are fluorine-free per se, in particular for the management of hernias, where the implant has at least in part threads having on their surface at least in part a fluorine-containing layer.

This disclosure relates to a medical implant which is suitable in particular for the management of hernias.

The management of a hernia or splanchnocele is one of the most important task areas in visceral or parietal surgery. This generally involves protrusion of intestines from the abdominal cavity through a congenital or acquired aperture, called the hernial orifice. The commonest forms of external hernias, where the hernial sac is always enveloped by the peritoneum, are inguinal, incisional and umbilical hernias. The causes for the occurrence of hernias are in particular weaknesses of muscles or connective tissues. These may arise as a result of overstrain, age-related slackening, inadequate scar formation following a surgical procedure or a congenital weakness of the abdominal wall.

A specific treatment involves in most cases a surgical procedure in which the contents of the hernia are replaced in the abdomen from the hernial sac, and the hernial orifice is closed. Closure of the hernial orifice can be carried out for example with the aid of suture materials. However, a disadvantage in this case is that suture materials can generally be exposed to mechanical stresses, like those occurring for example during coughing, to only a limited extent. Management of hernias solely with the aid of surgical suture materials therefore not uncommonly causes a recurrence of the hernia.

This is why there has been an increase in the use of artificial reinforcing materials in the form of textile meshes for reconstruction of the abdominal wall. These meshes have the advantage that they are able to take up forces in two or more directions and thereby have the effect of relieving the actual suture. Commercially available meshes predominantly consist of mono- or multifilament polymers, especially of polyester or polypropylene.

However, postoperative complications frequently occur when using hernia implants of the abovementioned type. These complications derive in particular from post-surgical adhesions to the abdominal cavity. This may permanently restrict the mobility of the patient. Such adhesions are also frequently painful for the patient. Overall, however, there is in particular an increased risk of a renewed surgical intervention.

This is why hernia implants which, owing to their special structure, are intended to prevent tissue adhesions, especially after an intraperitoneal implantation, are now commercially available. This can be achieved for example by an implant structure which is microporous on one side. It is possible by such a structure to substantially avoid cellular colonization and thus invasion of body cells on the microporous side of the implant. One example of such an implant is disclosed in DE 199 12 648 A1 and was developed by the present applicant.

The implants which are further employed are in particular composite implants which have coatings which are resistant to tissue adhesion on the side of the implant which is intended to face toward the abdominal cavity after the implantation. Hernia implants of this type are disclosed for example in EP 0 797 962 B1, EP 1 317 227 B1 and EP 0 998 314 B1. The coatings are frequently in the form of films or sheets. For example, a hernia mesh coated with a polymer sheet is described in DE 602 09 787 T2. However, films and sheets tend to have a certain fragility. This may eventually still lead to unwanted tissue infiltration of the implant, and thus the previously mentioned post-surgical tissue adhesions may occur.

EP 1 200 010 B1 describes hernia prosthesis with a barrier layer made of a fluoropolymer. The barrier material may be in particular polytetrafluoroethylene, fluorinated ethylene propylene, tetrafluoroethylene, ethylene tetrafluoroethylene and other suitable fluoropolymers. A further hernia implant with monofilament threads of polyvinylidene fluoride or a derivative derived therefrom is described in EP 1 411 997 B1. A disadvantage of an implant of this type is that its areal weight is generally higher than that for example of pure polypropylene meshes. In addition, the production of implants based on fluoropolymers is costly, attributable in particular to the high cost of the fluoromonomers used to prepare the fluoropolymers.

The object of this disclosure is therefore to provide a medical implant which is suitable in particular for intraperitoneal implantation without post-surgical tissue adhesions occurring in connection therewith. The implant is further intended to be able to be produced simply and, in particular, at low cost.

This object is achieved by a medical implant composed of polymers which are fluorine-free per se, in particular for the management of hernias, where the implant has at least in part threads having on their surface at least in part a fluorine-containing layer.

I provide an implant which, owing to its superficial fluorine-containing layer, has a hydrophobic nature. The implant thereby proves particularly advantageously to be resistant to possible post-surgical tissue adhesions, especially in the peritoneal region of the body.

The term “polymers which are fluorine-free” is intended to mean polymers which are prepared from fluorine-free monomers.

In a preferred embodiment, the thread surfaces, especially all implant threads, are surrounded or covered over the whole area by the fluorine-containing layer. Surrounding or coating the thread surfaces over the whole area with the fluorine-containing layer particularly advantageously increases the hydrophobic and thus the tissue adhesion-resistant nature of the implant.

Fluorine in the fluorine-containing layer is preferably covalently bonded to the polymer of the implant.

I further provide in particular for the polymers, apart from the fluorine-containing layer, to be free of fluorine.

It is further possible to provide for the implant to be a flat implant.

In a particularly preferred embodiment, the threads having the fluorine-containing layer are present on only one side face of the implant. This side is preferably the side face of the implant which faces toward the abdominal cavity after implantation.

It is possible in principle for the fluorine-containing layer to have a different layer thickness on the thread surfaces. However, the fluorine-containing layer on the thread surfaces preferably has a uniform layer thickness. It is possible in particular to provide for the fluorine-containing layer to have 1 mg of fluorine per m² of fluorinated thread surface. The fluorine-containing layer may penetrate into the implant in particular along a thin transitional layer.

In a further embodiment, the implant has an areal weight of between 36 and 70 g/cm². The implant preferably has an areal weight of between 36 and 45 g/cm², in particular 39 and 41 g/cm². In certain cases, especially with heavyweight patients, areal weights of between 45 and 70 g/cm², in particular 55 and 65 g/cm², may be advantageous. The areal weights mentioned in this section are advantageous inter alia because they make it possible for the implant still to have a certain flexibility, so that it can respond in an appropriate manner to the mechanical stresses occurring in the body after implantation thereof, for example tensile stresses caused by adjacent muscle tissue.

In a further embodiment, the implant has apertures with a clear width of the apertures of <6000 μm, in particular between 300 and 3500 μm. It is further preferred for the implant to have apertures with a total area which corresponds to 50 to 75%, in particular 58 to 70%, of the base area of the implant. The implant has in particular apertures which are free of the fluorine-containing layer. This means that the implant apertures in this embodiment are at least partly, preferably completely, not coated or covered by the fluorine-containing layer.

It is expedient for the fluorine-containing layer to be peel-resistantly connected to the thread surfaces. A peel-resistant connection means in this context that connection to the thread surfaces of the implant is as permanent as possible. This means in particular that the fluorine-containing layer does not become detached again from the implant threads after a certain time after an implantation and thus enters the surrounding tissue. The fluorine-containing layer is preferably connected by covalent bonds to the thread surfaces. The fluorine-containing layer may itself include a fluorine compound or consist of such a compound. It is preferred for at least some of the hydrogen atoms of the thread material in the fluorine-containing layer to be replaced by fluorine atoms. The fluorine-containing layer includes in particular fluorinated alkylene and/or alkyl groups. The alkylene or alkyl groups may be partly fluorinated and/or perfluorinated. The fluorinated alkylene or alkyl groups are preferably CHF—, CF₂—, CH₂F—, CHF₂— and/or CF₃— groups.

It may further be preferred for the implant to have a fluorine atom content of between 1 and 10% by weight, in particular 2 and 5% by weight, based on the total weight of the implant. The fluorine content, which is low per se, particularly advantageously does not bring about an increase in the weight of the implant which is disadvantageous in relation to mechanical or elastic properties.

The threads of the implant may be mono- and/or multifilament threads. The threads are preferably monofilament.

All fluorine-free polymers in principle come into consideration as suitable thread material. It is expedient for the polymers concerned to be biocompatible. The thread material is preferably a non-absorbable polymer. The thread material may be a polyolefin, in particular polyethylene and/or polypropylene. Further, polyesters, in particular polyethylenterephthalate, and/or polyamides may be used as thread materials.

The implant is in a preferred embodiment a textile implant, in particular a textile sheet-like structure. It is possible to provide for the implant to be a formed-loop knit, drawn-loop knit, braid, woven, web or scrim. The implant is preferably a formed-loop knit. It is provided in particular for the implant to be a textile mesh, in particular a hernia mesh, a prolapse mesh or a urinary incontinence mesh. In a particularly preferred embodiment, the implant is provided as a hernia mesh. The textile mesh is preferably a velour mesh. Concerning further details and features of the mesh, reference is made to the previous description.

In another embodiment, the implant is a suture material. Concerning further details and features of the suture material, reference is made to the previous description.

The implant particularly preferably includes sites engageable by body cells, in particular velour loops, floats and/or pile warps. It may further be provided for these engageable sites to be present on only one side, in particular side face, of the implant. The engageable sites may serve in particular as anchoring structures for the wall of the abdomen (abdominal wall).

It is also possible for the implant to be a three-dimensional textile product, in particular a plug. This three-dimensional textile product may be constructed for example from three or more sheet-like textile structures. The sheet-like textile structures can be connected, in particular welded, together at spots, in lines and/or over areas. I particularly provide for at least one sheet-like textile structure of the three-dimensional textile product to have a fluorine-containing layer.

This disclosure further relates to a process for producing the medical implant, where threads, and in particular threads of an implant, are at least partly treated with a fluorine gas mixture, by which means a fluorine-containing layer is generated on at least part of the thread surfaces. The fluorine gas mixture is a gas mixture which, besides fluorine, includes at least one further gas.

In a preferred embodiment, a gas mixture of fluorine and nitrogen, in particular in a fluorine/nitrogen ratio by volume of from 1/100 to 30/100, preferably 10/100, is used for treating the threads. The treatment of the threads is preferably carried out under a pressure of from 10³ Pa to 1.01325×10⁵ Pa (atmospheric pressure), in particular 10⁴ Pa to 5×10⁴ Pa. It may further be provided for the treatment of these threads to be carried out in a temperature range between 0° C. and 90° C., in particular 40° C. and 70° C. In a further embodiment, the treatment of the threads is carried out during a period of from 10 min to 240 min, in particular 25 min to 120 min.

It is possible where appropriate for a plurality of fluorine gas treatment cycles to be carried out in order, for example, to increase the layer thickness of the fluorine-containing layer formed on the thread surfaces by the treatment with the fluorine gas mixture.

The treated threads are further processed in a further embodiment to an implant, preferably textile implant. In this embodiment, the treated threads can be formed-loop knitted, woven, drawn-loop knitted or braided together. A further possibility is for the treated threads to be processed to a so-called nonwoven, especially a web or scrim. These textile processing techniques for threads are sufficiently well known to the skilled person for further statements to be omitted at this point.

As already mentioned, the threads are treated at least partly with a fluorine gas mixture. For this purpose, some of the threads, in particular the side face of an implant, can be covered with a suitable material, for example silicone. A treatment with fluorine gas mixture which is subsequently carried out leads merely to coating of the open, i.e. uncovered, threads.

This disclosure also relates to all medical implants which are produced or can be produced by a process.

This disclosure finally relates to the use of the medical implant in visceral and/or parietal surgery, in particular for the management of hernias.

Further details and features of my disclosure are evident from the following description of preferred embodiments in the form of examples in combination with the dependent claims. Individual features of my disclosure can be employed alone or in combination with other features in these embodiments. The preferred embodiments described are to be understood merely as descriptive disclosure which is not in any way limiting.

### EXAMPLE 1

### Fluorination of Optilene® Mesh LP

### Lightweight Mesh of Polypropylene Threads with an Areal Weight of about 38.1 g/m² and a Pore Size of About 1 mm

The Optilene® Mesh LP is cut to a size of about 36.5 cm×36.1 cm and then introduced into a fluorination reactor. Before the actual treatment of the mesh, two purging cycles with pure nitrogen in a pressure range between 10³ Pa and 8×10⁴ Pa are carried out. The mesh is then preheated under a nitrogen pressure of about 5×10⁴ Pa and during a period of about 300 seconds in the reactor. A fluorine gas mixture of fluorine and nitrogen, which may where appropriate also contain technical nitrogen, is used to fluorinate the mesh. The proportion of fluorine in the treatment gas is about 10% by volume. The fluorination of the mesh is carried out under a gas pressure of about 3.5×10⁴ Pa and during a period of about 3600 seconds. Two purging cycles are then carried out in order to remove remaining gases from the reactor.

The treated mesh showed purely externally no changes, in particular no changes in color. Weighing of the treated mesh revealed a slight increase in weight of about 4.01% compared with the untreated mesh. This shows that the lightweightness of the mesh is substantially unaffected by the fluorination.

### EXAMPLE 2

### Production of an Optilene® Mesh LP Which is Fluorinated Only on a Side Face

A lightweight Optilene® Mesh LP (cf. example 1) is suspended in a fluorination reactor as described in example 1, with one side face of the mesh being covered by a silicone mat. The mesh is then fluorinated in accordance with example 1. After the fluorination treatment is complete, the silicone mat is removed and the mesh is washed with a solvent mixture of 2-propanol and acetone (2-propanol/acetone 70/30 (v/v)). The mesh obtained in this way is fluorinated only on its uncovered side face.

### EXAMPLE 3

### Animal Experimental Investigation

Eight female SPF albino rabbits of the Chbb:HM(SPF) Kleinrusse breed (Charles River Deutschland GmbH) were used to carry out the animal experiments. The rabbits had a body weight of between 2.4 and 3.2 kg and were provided with National Wing Band earmarks. To prepare the implantation, the rabbits were anesthetized and then their cecum was traumatized. The cecum was subsequently traumatized by means of a sterile gauze compress for about 3.5 minutes during which spots and patches of slight bleeding appeared on the surface of the cecum. Subsequently, a piece about 2.5 cm×2.5 cm in size of the peritoneum with the underlying transverse abdominal muscle was removed from the right side of the abdominal wall. A piece of mesh of an appropriate size (3.5 cm×3.5 cm) was placed on the abdominal wall defect and fixed with an encircling suture (Resopren® suture material). An Optilene® mesh treated as in example 1 was implanted in this way into four rabbits. The other four rabbits received an untreated lightweight Optilene® mesh. The animals were sacrificed 21 days after the operation, necropsied and subjected to gross assessment of the proportion of the explanted meshes which showed adhesion. The results which emerged in this case are as follows:

| | | Adhesions as % of |
|---|---|---|
| | | the total area of the |
| | Animal No. | abdominal wall defect |
| Mesh | 3091 | 30 |
| (treated in | 3097 | free |
| accordance with | 3123 | 40 |
| example 1) | 3165 | free |
| Mesh | 3185 | 90 |
| (untreated) | 3391 | 85 |
| | 3352 | 100 |
| | 3415 | 95 |

The results clearly show that the fluorinated meshes bring about a significant reduction in post-surgical tissue adhesions compared with the unfluorinated meshes. Two of the explanted fluorinated hernia meshes showed absolutely no post-surgical tissue adhesions.

1. A medical implant composed of fluorine-free polymers which are for management of hernias, wherein the implant has at least in part threads having on their surface at least in part a fluorine-containing layer.

2. The implant as claimed in claim 1, wherein the thread surfaces are entirely surrounded by the fluorine-containing layer.

3. The implant as claimed in claim 1, wherein the fluorine-containing layer is covalently bonded to the polymer of the implant.

4. The implant as claimed in claim 1, wherein the polymers, apart from the fluorine-containing layer, are free of fluorine.

5. The implant as claimed in claim 1, wherein that the threads having the fluorine-containing layer are present on only one side face of the implant.

6. The implant as claimed in claim 1, wherein the fluorine-containing layer on the thread surfaces has a uniform layer thickness.

7. The implant as claimed in claim 1, wherein the fluorine-containing layer has 1 mg of fluorine per m² of fluorinated thread surface.

8. The implant as claimed in claim 1, wherein the implant has an areal weight of between 36 and 70 g/cm².

9. The implant as claimed in claim 1, wherein the implant has apertures with a clear width of the apertures of <6000 μm.

10. The implant as claimed in claim 1, wherein the implant has apertures with a total area which corresponds to 50 to 75% of the base area of the implant.

11. The implant as claimed in claim 1, wherein the implant has apertures which are free of the fluorine-containing layer.

12. The implant as claimed in claim 1, wherein the fluorine-containing layer is peel-resistantly connected to the thread surfaces by covalent bonds.

13. The implant as claimed in claim 1, wherein the fluorine-containing layer has fluorinated alkylene and/or alkyl groups.

14. The implant as claimed in claim 1, wherein the fluorine-containing layer has CHF—, CF₂—, CH₂F—, CHF₂— and/or CF₃— groups.

15. The implant as claimed in claim 1, wherein the implant has a fluorine atom content of between 1 and 10% by weight, based on the total weight of the implant.

16. The implant as claimed in claim 1, wherein the thread material is a non-absorbable polymer.

17. The implant as claimed in claim 1, wherein the thread material is a polyolefin.

18. The implant as claimed in claim 1, wherein the thread material is at least one selected from the group consisting of polyethylene and polypropylene.

19. The implant as claimed in claim 1, wherein the implant is a textile implant.

20. The implant as claimed in claim 1, wherein the implant is a textile mesh.

21. The implant as claimed in claim 1, wherein the implant is a suture material.

22. The implant as claimed in claim 1, wherein the implant has sites engageable by body cells.

23. The implant as claimed in claim 1, wherein the implant has at least one selected from the group consisting of velour loops, floats and pile warps.

24. A process for producing the implant as claimed in claim 1 comprising treating threads at least partly with a fluorine gas mixture to thereby generate a fluorine-containing layer on at least a part of the thread surfaces.

25. The process as claimed in claim 24, wherein a gas mixture of fluorine and nitrogen in a fluorine/nitrogen ratio by volume of 1/100 to 30/100 treats the threads.

26. The process as claimed in claim 24, wherein treatment of the threads is carried out under a pressure of from 10³ Pa to 1.01325×10⁵ Pa.

27. The process as claimed in claim 24, wherein treatment of the threads is carried out in a temperature range between 0° C. to 90° C.

28. The process as claimed in claim 24, wherein treatment of the threads is carried out during a period of from 10 to 240 min.

29. (canceled)

30. A medical implant produced by a process as claimed in claim 24.

31. A process for visceral and/or parietal surgery for the management of hernias comprising applying a medical implant as claimed in claim 1 to a patient.
